(19) United States
(12) Patent Application Publication
(10) Pub. No.: US 2015/0080437 A1
(43) Pub. Date: Mar. 19, 2015

(54) TOPICAL ADHESIVE COMPOSITION, AND DEVICE, FOR IMPROVING AESTHETIC APPEARANCE OF SKIN

(71) Applicant: CORIUM INTERNATIONAL, INC., Menlo Park, CA (US)

(72) Inventors: Eun Soo Lee, Redwood City, CA (US); Appala Sagi, Mountain View, CA (US); Parminder Singh, Union City, CA (US)

(21) Appl. No.: 14/489,181

(22) Filed: Sep. 17, 2014

Related U.S. Application Data
(60) Provisional application No. 62/024,079, filed on Jul. 14, 2014.
(60) Provisional application No. 61/879,031, filed on Sep. 17, 2013.

(51) Int. Cl. A61K 8/49; A61Q 19/08; A61K 8/81

(52) U.S. Cl. CPC A61K 8/494; A61K 8/8176; A61K 8/8147; A61Q 19/08; A61K 2800/546
USPC 514/356

(57) ABSTRACT

Compositions, devices and methods for improving the aesthetic appearance of skin are described. In one embodiment, the compositions, devices and methods reduce fine lines and/or wrinkles, and in other embodiment, the compositions, devices and methods improve skin tone by reducing regions of hyperpigmentation. The cosmetic compositions and devices described are formulated for topical application for extended wear, and comprise one or more agents to achieve the desired beneficial effect. The composition is preferably an adhesive composition that is secured to the skin for an extended time for sustained release of the one or more beneficial agents.

### CROSS-REFERENCE TO RELATED APPLICATIONS

This application claims the benefit of U.S. Provisional Application No. 62,024,079, filed Jul. 14, 2014 and of U.S. Provisional Application No. 61/879,031, filed Sep. 17, 2013, each of which is incorporated by reference herein.

### TECHNICAL FIELD

The subject matter described herein relates to compositions and methods for improving aesthetic appearance of skin. The compositions are topically applied, and worn for an extended period of time, to deliver one or more active agents that beneficially alters the skin's appearance, such as by attenuating lines and wrinkles on skin, reducing redness associated with acne lesions, reducing pigmentation associated with age spots and hyperpigmentation, or improving an uneven skin tone by diminishing areas of hyperpigmentation and/or hypopigmentation.

### BACKGROUND

Aging and chronic exposure to adverse environmental factors alters the visual appearance, physical properties, and physiological functions of skin in ways that are, unfortunately, considered visually undesirable. Notable changes are the development of fine lines and wrinkles, loss of elasticity, increased sagging, loss of firmness, loss of color evenness or tone and mottled pigmentation. Many of the alterations in appearance and function of the skin are caused by changes in the outer epidermal layer of the skin, while others are caused by changes in the lower dermis.

The effectiveness of all skin care products is normally contingent upon delivery of the active ingredients therein through the stratum corneum and viable epidermis into the dermis layer of the skin structure. This is because the active ingredients in the skin care product cannot be effective unless they penetrate through the dead layers of skin tissue and into the dermis layer of living skin cells. This is normally a difficult proposition for water soluble active ingredients because the stratum corneum is a good water barrier. The stratum corneum and viable epidermis act to protect the body by holding water therein to prevent dehydration and by keeping external water which is frequently contaminated out of the body.

There is a demand for cosmetic compositions and cosmetic methods for improving the appearance and condition of skin. Consumers seek “anti-aging” cosmetic products that treat or delay the visible signs of actual aging and weathered skin, such as wrinkles, lines, sagging, hyperpigmentation and age spots. Consumers also seek other benefits from cosmetic products in addition to anti-aging benefits. For example, the concept of “sensitive skin” has raised the demand for cosmetic products that improve the appearance and condition of sensitive, dry and/or flaky skin, and that soothe red, and/or irritated skin. Consumers also desire cosmetic products that treat acne and other skin blemishes.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

It is an object of the present invention to provide methods and compositions to effect at least a temporary physiological improvement in the condition of the skin, with the desired goal of a lasting and permanent improvement and enhancement of the condition and quality of the skin.

It is a related object of the invention to improve, diminish or alleviate fine lines and/or wrinkles, including fine wrinkles, on the face, especially around the eyes, around the upper and lower lips, and in the smaller area of the cheeks, such fine lines and/or wrinkles being physical signs associated with intrinsic or extrinsic dermatological aging.

It is a related object of the invention to improve, diminish or attenuate redness associated with acne, especially acne on the face.

It is related object of the invention to improve skin tone, by reducing regions of hyperpigmentation such as those often referred to as age spots and such as regions of discoloration associated with aging skin's exposure to sun.

It is another object of the present invention to provide improved, safe and inexpensive therapeutic and/or cosmetic compositions for the treatment of various skin conditions, and in one embodiment for the treatment of fine lines and/or wrinkles, including fine wrinkles, of the facial area, without the concomitant skin dryness, scaling, chafing, burning, stinging and irritation and/or other disadvantages that are associated with other types of anti-wrinkle and age-related skin condition treatment compositions and methods.

In one aspect, a topical skin composition for improving the visual appearance of skin is provided. The composition comprises a skin-contacting layer comprised of (i) an adhesive mixture comprised of a water-swellable, water-insoluble film forming polymer and a water-soluble film forming polymer in a ratio of between about 0.2-0.7; (ii) between about 15-55 wt % of a solvent mixture comprised of water, a polyol and an alpha hydroxyl acid ester or fatty alcohol; and (iii) between about 1-50 wt % of an active agent capable of improving the visual appearance of skin.

In another aspect, a topical skin composition for reducing the visual appearance of a fine line or a wrinkle is provided. The composition comprises a skin-contacting layer comprised of (i) an adhesive mixture comprised of a water-swellable, water-insoluble film forming polymer and a water-soluble film forming polymer in a ratio of between about 0.2-0.7; (ii) between about 15-55 wt % of a solvent mixture comprised of water, a polyol and an alpha hydroxyl acid ester or fatty alcohol; and (iii) between about 1-50 wt % of an active agent capable of reducing visual appearance of fine lines and/or wrinkles.

In another aspect, a topical skin composition for improving skin tone is provided. The composition comprises a skin-contacting layer comprised of (i) an adhesive mixture comprised of a water-swellable, water-insoluble film forming polymer and a water-soluble film forming polymer in a ratio of between about 0.2-0.7; (ii) between about 15-55 wt % of a solvent mixture comprised of water, a polyol and an alpha hydroxyl acid ester or fatty alcohol; and (iii) between about 1-50 wt % of an active agent capable of reducing regions of hyperpigmentation such as those often referred to as age spots and such as regions of discoloration associated with aging skin's exposure to sun.

In another aspect, a topical skin composition for treating existing acne lesions, reducing redness associated with acne lesions and/or protecting from formation of acne lesions is provided. The composition comprises a skin-contacting layer comprised of (i) an adhesive mixture comprised of a water-swellable, water-insoluble film forming polymer and a water-soluble film forming polymer in a ratio of between about 0.2-0.7; (ii) between about 15-55 wt % of a solvent mixture comprised of water, a polyol and an alpha hydroxyl acid ester or fatty alcohol; and (iii) between about 1-50 wt % of an active agent capable of treating existing acne lesions, reducing redness associated with acne lesions and/or protecting from formation of acne lesions.

In another aspect, a device for topical application to the skin is described. The device comprises a backing layer and a skin-contacting layer deposited on the backing layer, the skin-contacting layer comprised of (i) an adhesive mixture comprised of a water-swellable, water-insoluble film forming polymer and a water-soluble film forming polymer in a ratio of between about 0.2-0.7; (ii) between about 15-55 wt % of a solvent mixture comprised of water, a polyol and an alpha hydroxyl acid ester or fatty alcohol, the solvent mixture in liquid form at room temperature; and (iii) between about 1-50 wt % of an active agent.

In one embodiment, the adhesive mixture further comprises an oligomer capable of hydrogen or electrostatic bonding to the water-soluble film forming polymer.

In another embodiment, wherein the water-soluble film forming polymer and the oligomer form a homogenous blend.

In another embodiment, the water-swellable, water-insoluble film forming polymer is a cellulose ester or an acrylate polymer.

In still another embodiment, the acrylate polymer is selected from polymers and copolymers of acrylic acid, methacrylic acid, methyl acrylate, ethyl acrylate, methyl methacrylate, or ethyl methacrylate.

In yet another embodiment, the water-soluble film forming polymer is selected from poly(N-vinyllactams), poly(N-vinyl amides), poly(N-alkylacrylamides), polyacrylic acids, polymethacrylic acids, polyvinyl alcohol, polyvinylamine, and copolymers and blends thereof.

In another embodiment, the oligomer is selected from polyhydric alcohols, monomeric and oligomeric alkylene glycols, polyalkylene glycols, carboxyl-terminated polyalkylene glycols, amino-terminated polyalkylene glycols, ether alcohols, alkane diols and carbonic diacids.

In still another embodiment, a hydrophobic adhesive layer is disposed between the backing layer and the adhesive layer.

In various embodiments, the active agent is selected from niacinamide, alone or in combination with one or more of palmitoyl-lysine-threonine, palmitoyl-lysine-threonine-threonine-lysine-serine, N-undecyl-10-enoyl-L-phenylalanine, retinyl propionate, N-acetyl glucosamine, vitamin C, tretinoin, salicylic acid, benzoic acid, benzoyl peroxide, tretinoin, and combinations thereof.

In yet another aspect, a method for reducing fine lines and/or wrinkles on human skin is provided, and comprises topically applying an adhesive composition or device as described herein.

In yet another aspect, a method for treating existing acne lesions, reducing redness associated with acne lesions and/or protecting from formation of acne lesions is provided is provided, and comprises topically applying an adhesive composition or device as described herein.

In yet another aspect, a method for improving skin tone, such as by reducing regions of hyperpigmentation such as those often referred to as age spots or by reducing regions of discoloration associated with aging skin's exposure to sun is provided, and comprises topically applying an adhesive composition or device as described herein.

In other aspects, methods for improving the visual appearance of skin include increasing the hydration of skin by topically applying an adhesive composition or device as described herein and wearing the composition or device for a period of at least about 4 hours. In one embodiment, the composition or device comprises a backing layer or other layer that renders it occlusive to transmission of water vapor, thus increasing the water content of the skin in contact with the device or composition, to increase skin hydration content relative to the skin hydration content prior to application of the device or composition. Improved hydration of the skin diminishes the appearance of fine lines and wrinkles.

In other aspects, methods for improving the visual appearance of skin include increasing the smoothness, brightness and/or texture of the skin by topically applying an adhesive composition or device as described herein and wearing the composition or device for a period of at least about 4 hours.

In one embodiment, the methods further comprise wearing the composition or device for a period of at least about 4 hours, 6 hours, 8 hours, 10 hours, or 12 hours. In still another embodiment, the composition or device is worn while sleeping. In yet another embodiment, the composition or device is topically applied to the face. In a particular embodiment, the composition or device is topically applied under and/or around the eyes.

In one embodiment, the methods further comprise applying and wearing a device or composition as described herein daily, every other day, every third day, every fourth day, every sixth day, or once weekly. On days when a device or composition is not applied and being worn for a period of time, a different treatment regimen is applied or conducted on the skin. The different treatment regimen may be application of a cosmetic product, such as a cream or ointment, conducting a non-surgical cosmetic procedure, such as a light or laser treatment, a multi-level skin peel, microdermabrasion, ultrasound or microneedling.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B show the in vitro flux, in μg/cm²·hr (FIG. 1A), and the cumulative permeation in μg/cm² (FIG. 1B), of niacinamide through human cadaver skin from a device as described herein, where the adhesive layer in the devices comprised 10 wt % (larger squares) or 15 wt % (triangles) of niacinamide in the adhesive layer; release of niacinamide from a reference device is also shown (smaller squares);

FIGS. 2A-2B show the in vitro flux, in μg/cm²·hr (FIG. 2A), and the cumulative permeation in μg/cm² (FIG. 2B), of niacinamide through human cadaver skin from a device as described herein, as a function of time (hours), where the adhesive layer in the devices comprised lauryl lactate (squares) or triacetin (triangles) in the adhesive layer;

FIGS. 3A-3B show the in vitro flux, in μg/cm²·hr (FIG. 3A), and the cumulative permeation in μg/cm² (FIG. 3B), of niacinamide through human cadaver skin from a device as described herein, as a function of time (hours), where the adhesive layer in the devices comprised 18.52 wt % water (larger squares), 17.60 wt % water (triangles), 16.60 wt % water (smaller squares) in the adhesive layer; release of niacinamide from a reference device is also shown (smaller triangles);

FIGS. 4A-4B show the in vitro flux, in μg/cm²·hr (FIG. 4A), and the cumulative permeation in μg/cm² (FIG. 4B), of niacinamide through human cadaver skin from a device as described herein, as a function of time (hours), where the backing layer in the devices was a 4 mil EVA film (larger squares), a 3 mil PE film (diamonds), a 4 mil EVA film with a VA content different from the other backing film (smaller squares); release of niacinamide from a reference device is also shown (smaller triangles);

FIGS. 5A-5B show the in vitro flux, in μg/cm²·hr (FIG. 5A), and the cumulative permeation in μg/cm² (FIG. 5B), of niacinamide through human cadaver skin with a high skin permeability, from a device as described herein with an adhesive composition as set forth in Example 1, as a function of time in hours (squares); release of niacinamide from a reference device is also shown (diamonds);

FIGS. 6A-6B show the in vitro flux, in μg/cm²·hr (FIG. 6A), and the cumulative permeation in μg/cm² (FIG. 6B), of niacinamide through human cadaver skin with a high skin permeability, from a device as described herein with an adhesive composition as set forth in Example 1, as a function of time in hours (squares); release of niacinamide from a reference device is also shown (diamonds); and

FIGS. 7A-7B show the in vitro flux, in μg/cm²·hr (FIG. 7A), and the cumulative permeation in μg/cm² (FIG. 7B), of niacinamide through human cadaver skin with a low skin permeability, from a device as described herein with an adhesive composition as set forth in Example 1, as a function of time in hours (squares); release of niacinamide from a reference device is also shown (diamonds).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

“Improving the appearance of skin” and “improving the aesthetic appearance of skin” are used interchangeably herein to designate an aesthetic improvement in the appearance of skin. Representative improvements may include, but are not limited to, favorable characteristics and/or properties related skin thickness, elasticity, resiliency, smoothness, tone, texture, brightness, clarity, contour, firmness, tautness, and/or suppleness, and/or combinations thereof. In one embodiment, the terms intend the appearance of facial skin.

The term “effective amount” means an amount sufficient to cause a cosmetic effect to the skin.

### II. Composition/Device

The composition, and device, described herein are designed for topical application to the skin of a human, to improve, diminish or alleviate fine lines and/or wrinkles, in particular on the face, especially around (including under and at the outer edges where so-called “Crow's feet” form) the eyes, around the upper and lower lips, and in the smaller area of the cheeks. The fine lines and/or wrinkles are physical signs associated with intrinsic or extrinsic dermatological aging, and their appearance can be reduced by the composition, device and method described herein. The composition is intended for use by humans of either gender and any race or ethnicity, and provides improved appearance by diminishing fine line and wrinkles on normal healthy or diseased skin.

The composition comprises an adhesive layer intended for contact, and adhesion, to the skin. The composition described herein provides sufficient adhesion to the skin for wearing the composition, or a device comprising the composition, comfortably for a period of wear of, for example, 2-12 hours, 4-12 hours, 4-8 hours, 4-6 hours, or 3-9 hours, or for at least about 2 hours, 3 hours, 4 hours, 5 hours, 6 hours, 7 hours or 8 hours. After the period of wear, the composition is removed from the skin with no or negligible residue of the adhesive layer on the skin. As will be described, the composition comprises a significant fraction by weight of liquid or solvent, and as skilled artisans will appreciate, adhesive matrices with significant fractions of liquid suffer from poor cohesion and adhesion, as well as adhesive residue remaining on the skin upon removal. The composition described herein provides good cohesion, adhesion and little to no residue upon removal.

In one embodiment, the composition comprises a skin-contacting adhesive matrix or layer. The adhesive matrix is comprised of (i) an adhesive mixture comprised of a water-swellable, water-insoluble film forming polymer and a water-soluble film forming polymer; (ii) a solvent mixture comprised of water, a polyol and an alpha hydroxyl acid ester or fatty alcohol; and (iii) an active agent capable of reducing visual appearance of fine lines and/or wrinkles.

The adhesive mixture comprises at least two polymers, the first polymer a water-swellable, water-insoluble polymer and the second polymer a water-soluble polymer. The term “water-insoluble” refers to a compound or composition whose solubility in water is less than 5 wt %, preferably less than 3 wt %, more preferably less than 1 wt % (measured in water at 20° C.). In a preferred embodiment, both the water-insoluble polymer and the water-soluble polymer are film forming polymers, which refers to polymer that produce a physical, continuous and flexible film upon ‘dry-down’ or evaporation of a casting solvent. Studies performed in support of the claimed composition demonstrate that a composition wherein the ratio of water-insoluble polymer to water-soluble polymer is between about 0.2-0.7 yields a composition capable of accommodating a significant weight percent of solvent mixture (described below) with good adhesivity and cohesivity, and no or negligible residue upon removal from skin. In other embodiments, the ratio of water-insoluble polymer to water-soluble polymer is between about 0.1-1.0, 0.25-0.65, 0.3-0.60. 0.3-0.58, or 0.3-0.57.

In one embodiment, the water-insoluble polymer is water-swellable and film forming. Exemplary polymers include cellulose esters, alginic acid, and acrylate polymers. The cellulose ester can be comprised of at least one cellulosic polymer containing unesterified cellulose monomer units, cellulose acetate monomer units, and either cellulose butyrate monomer units or cellulose propionate monomer units. Exemplary cellulose esters are, for example, cellulose acetate, cellulose acetate propionate, cellulose acetate butyrate, cellulose propionate, cellulose butyrate, cellulose propionate butyrate, cellulose diacetate, and cellulose triacetate. Such cellulose esters typically have a number average molecular weight of between about 10,000 and about 75,000.

Generally, the cellulose ester comprises a mixture of cellulose and cellulose ester monomer units; for example, commercially available cellulose acetate butyrate contains cellulose acetate monomer units as well as cellulose butyrate monomer units and unesterified cellulose monomer units, while cellulose acetate proprionate contains monomer units such as cellulose proprionate. Preferred cellulose esters herein are cellulose acetate propionate compositions and cellulose acetate butyrate.

When the water-insoluble polymer is an acrylate, it may be selected from polymers and copolymers of acrylic acid, methacrylic acid, methyl acrylate, ethyl acrylate, methyl methacrylate, or ethyl methacrylate. The term “acrylate polymer” is intended to include acrylate and acrylate-based polymers and copolymers, and is an acrylic acid or acrylic acid ester polymer. Suitable acrylate polymers are those copolymers available under the tradename EUDRAGIT® from Rohm Pharma (Germany). Preferred acrylate polymers are copolymers of methacrylic acid and methyl methacrylate, such as the EUDRAGIT® L and EUDRAGIT® S series polymers. Particularly preferred such copolymers are EUDRAGIT® L 30D-55 and EUDRAGIT® L 100-55 (the latter copolymer is a spray-dried form of EUDRAGIT® L 30D-55 that can be reconstituted with water). The molecular weight of the EUDRAGIT® L 30D-55 and EUDRAGIT® L 100-55 copolymer is approximately 135,000 Da, with a ratio of free carboxyl groups to ester groups of approximately 1:1. The EUDRAGIT® L 100-55 copolymer is generally insoluble in aqueous fluids having a pH below 5.5. Another exemplary methacrylic acid-methyl methacrylate copolymer is EUDRAGIT® S-100, which differs from EUDRAGIT® L 30D-55 in that the ratio of free carboxyl groups to ester groups is approximately 1:2. It will be appreciated by those skilled in the art that EUDRAGIT® L 30D-55, L 100-55, L 100, and S 100 are exemplary, and other acceptable polymers having similar pH-dependent solubility characteristics can be used.

The water-soluble polymer is a hydrophilic polymer that typically includes repeating units derived from an N-vinyl lactam monomer, a carboxy vinyl monomer, a vinyl ester monomer, an ester of a carboxy vinyl monomer, a vinyl amide monomer, and/or a hydroxy vinyl monomer. Such polymers include, by way of example, poly(N-vinyl lactams), poly(N-vinyl acrylamides), poly(N-alkylacrylamides), substituted and unsubstituted acrylic and methacrylic acid polymers (e.g., polyacrylic acids and polymethacrylic acids), polyvinyl alcohol (PVA), polyvinylamine, copolymers thereof and copolymers with other types of hydrophilic monomers (e.g. vinyl acetate).

Poly(N-vinyl lactams) are preferably non-crosslinked homopolymers or copolymers of N-vinyl lactam monomer units, with N-vinyl lactam monomer units representing the majority of the total monomeric units of a poly(N-vinyl lactams) copolymer. Preferred poly(N-vinyl lactams) are prepared by polymerization of one or more of the following N-vinyl lactam monomers: N-vinyl-2-pyrrolidone; N-vinyl-2-valerolactam; and N-vinyl-2-caprolactam. Nonlimiting examples of non-N-vinyl lactam comonomers useful with N-vinyl lactam monomeric units include N,N-dimethylacrylamide, acrylic acid, methacrylic acid, hydroxyethylmethacrylate, acrylamide, 2-acrylamido-2-methyl-1-propane sulfonic acid or its salt, and vinyl acetate. Poly (N-alkylacrylamides) include, by way of example, poly(methacrylamide) and poly(N-isopropyl acrylamide) (PNIPAM). Preferred hydrophilic polymers herein are the following: poly(N-vinyl lactams), particularly polyvinyl pyrrolidone (PVP) and polyvinyl caprolactam (PVCap); and poly(N-vinyl acetamides).

The molecular weight of the hydrophilic polymer is not critical; however, the number average molecular weight of the hydrophilic polymer is generally in the range of approximately 100,000 to 2,000,000, more typically in the range of approximately 500,000 to 1,500,000.

In some embodiments, the polymer mixture in the adhesive layer additionally comprises an oligomeric polymer, sometimes referred to herein as an oligomer. The oligomer is “complementary” to the hydrophilic polymers in that it is capable of hydrogen or electrostatic bonding, thereto. Preferably, the complementary oligomer is terminated with hydroxyl groups, amino or carboxyl groups. The oligomer typically has a glass transition temperature Tg in the range of about −100° C. to about −30° C. and a melting temperature Tm lower than about 20° C. The oligomer may be also amorphous. The difference between the Tg values of the hydrophilic polymer and the oligomer is preferably greater than about 50° C., more preferably greater than about 100° C., and most preferably in the range of about 150° C. to about 300° C. The hydrophilic polymer and complementary oligomer should be compatible, i.e. capable of forming a homogeneous blend.

As noted above, the complementary oligomer is capable of hydrogen or electrostatic bonding to the hydrophilic polymer. The complementary oligomer may be capable of covalently bonding to the hydrophilic polymer as well. In addition, the complementary oligomer may be capable of hydrogen or electrostatic bonding to the water-swellable, water-insoluble polymer.

Generally, the complementary oligomer will have a molecular weight in the range from about 45 to about 800, preferably in the range of about 45 to about 600. The complementary oligomer is preferably a low molecular weight polyalkylene glycol (molecular weight 200-600) such as polyethylene glycol 400, which can also serve as a low molecular weight plasticizer. Alternatively, a different compound can be incorporated as an additional low molecular weight plasticizer, in which case any of the low molecular weight plasticizers described below can be used. In one embodiment, the complementary oligomer is a complementary low molecular weight or oligomeric plasticizer that contains at least two functional groups per molecule that are capable of hydrogen or electrostatic bonding to the hydrophilic polymer.

Examples of suitable complementary oligomers include, but are not limited to, low molecular weight polyhydric alcohols (e.g. glycerol or sorbitol), monomeric and oligoalkylene glycols such as ethylene glycol and propylene glycol, ether alcohols (e.g., glycol ethers), carbonic diacids, alkane diols from butane diol to octane diol, including carboxyl-terminated and amino-terminated derivatives of polyalkylene glycols. Polyalkylene glycols, optionally carboxyl-terminated, are preferred herein, and polyethylene glycol having a molecular weight in the range of about 200 to 600 is an optimal complementary oligomer. It will be appreciated from the foregoing that a single compound, e.g., a low molecular weight polyalkylene glycol such as polyethylene glycol having a molecular weight in the range of about 200 to 600, can serve as both the complementary oligomer and the low molecular weight plasticizer.

The adhesive mixture components, and methods of mixing them, are described, for example in U.S. Pat. No. 8,206,738, RE44,145, and U.S. Pat. No. 8,481,059, which are each incorporated by reference herein in their entireties.

The adhesive layer in the composition also comprises a solvent component, typically in an amount of between about 15-55 wt % of the total mass of the adhesive layer. In one embodiment, the solvent component is comprised of water and a polyol, and in another embodiment, the solvent component additionally comprises an alpha hydroxyl acid ester or fatty alcohol. In another embodiment, the solvent component additionally comprises an humectant, emollient or moisturizer, such as panthenol.

The polyol is in one embodiment a sugar alcohol, and in one embodiment is a monomeric polyol, such as glycerin (glycerol), pentaerythritol, or ethylene glycol. The polyol is typically present in the adhesive composition between 1-20 wt %, preferably between 3-20 wt %, more preferably between 6-20 wt %.

Alpha hydroxyl acid ester and fatty alcohols are known to skilled artisans, and are described, for example in EP 0831767, which is incorporated herein by reference. In the examples set forth herein, the alpha hydroxyl acid ester is lauryl lactate, and it will be appreciated that this is merely exemplary. The alpha hydroxyl acid ester or fatty alcohol is typically present in the adhesive composition between 0-3 wt %, preferably between 0.1-2.5 wt %, more preferably between 0.1-2 wt %.

In one embodiment, the weight percent of water, polyol and alpha hydroxyl acid ester or fatty alcohol in the adhesive composition is between about 10-60 wt %, preferably between 15-55 wt %, more preferably between 17-54 wt %.

The adhesive layer additionally comprises an agent having activity to improve visual or aesthetic appearance of the skin, such as an agent effective to reduce or diminish the appearance of fine lines and/or wrinkles on human facial skin or an agent effective to treating existing acne lesions, reducing redness associated with acne lesions and/or protecting from formation of acne lesions. Exemplary agents are described below.

Vitamins: In various embodiments the adhesive layer of the present invention may comprise one or more vitamins. Herein, “vitamins” means vitamins, pro-vitamins, and their salts, isomers and derivatives. The vitamins may include those vitamins not known to exhibit significant antioxidant properties, for example, vitamin D compounds; vitamin K compounds; and mixtures thereof. The compositions of the present invention optionally may include those which exhibit antioxidant properties, non-limiting examples of suitable vitamins include: vitamin B compounds (including niacinamide, nicotinic acid, C1-C18 nicotinic acid esters, and nicotinyl alcohol; B6 compounds, such as pyroxidine; and B5 compounds, such as panthenol, or “pro-B5”); vitamin A compounds, and all natural and/or synthetic analogs of Vitamin A, including retinoids such as retinyl propionate, carotenoids, and other compounds which possess the biological activity of Vitamin A; vitamin E compounds, or tocopherol, including tocopherol sorbate, tocopherol acetate, other esters of tocopherol; vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate. In one embodiment, the compositions of the instant invention may comprise from about 0.0001% to about 50%, alternatively from about 0.001% to about 10%, alternatively from about 0.01% to about 5%, and alternatively from about 0.1% to about 1%, of the vitamin.

Peptides and Peptide Derivatives: The adhesive layer may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (pal-KTTKS, available in a composition known as MATRIXYL®) palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®).

In various embodiments the adhesive layer may comprise from about 1×10⁻⁷% to about 20%, alternatively from about 1×10⁻⁶% to about 10%, and alternatively from about 1×10⁻⁵% to about 5% of the peptide.

Oil Control Agents: In various embodiments the compositions may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

Other Skin Care Agents: The compositions in various embodiments may comprise N-acyl amino acid compounds. Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE (Registered trademark) from Seppic (France).

Skin care agents are disclosed in US Publication No. 2007/0020220A1, published Jan. 25, 2007, wherein the components/ingredients are incorporated herein by reference in their entirety.

In one embodiment, the agent is niacinamide. In one embodiment, the agent is a combination of niacinamide, glycerine, tocopherol acetate, and/or D-panthenol. Niacinamide may be included in the composition in an amount between 1-30 wt %, preferably between 2-28 wt %, more preferably between 5-25 w %, still more preferably between 10-20 wt %. When D-panthenol is included, it may be present in an amount between 0.5-5 wt %, or between 0.5-3 wt % or 0.5-2 wt %. Glycerin may be included as an active in an amount between 6-20 wt %, 8-15 wt %, 10-15 wt %. Tocopherol acetate, if included, may be present in an amount between 0.1-10 wt %, preferably between 0.2-8 wt %. The amount of active in the adhesive composition, whether as a single agent or more than one agent, can be between 1-50 wt %, 2-50 wt %, 5-50 wt %, 5-30 wt %, 5-25 wt %, 10-50 wt %, 10-30 wt %, 10-25 wt %, 10-20 wt %, 15-50 wt %, or 15-30 wt %.

In one embodiment, the agent with activity to reduce or diminish the appearance of fine lines and/or wrinkles on human skin is soluble in the adhesive mixture, and in another embodiment, the agent at the wt % included in the adhesive mixture is soluble therein.

The adhesive layer may additionally comprise antioxidants and/or preservatives and/or cosmetically acceptable excipients, such as, but not limited to, vitamin E, vitamin E acetate and methyl paraben.

Table 1 presents exemplary compositions and a contemplated exemplary range for each component in the exemplary compositions and devices described herein.

TABLE 1
Exemplary Compositions

| | #1 | #2 | #3 | #4 | Range |
|---|---|---|---|---|---|
| Component | (wt %) | (wt %) | (wt %) | (wt %) | (wt %) |
| Water-soluble | 25.30% | 25.30% | 35% | 23% | 20%-45% |
| polymer | | | | | |
| Oligomer | 12.65% | 12.65% | 18% | 18% | 6%-20% |
| Water-insoluble | 12.65% | 12.65% | 8% | 15% | 5%-20% |
| polymer | | | | | |
| Glycerin | 15% | 15% | 11% | 18.6% | 6%-20% |
| Water | 29.3% | 26.3% | 9.6% | 18% | 10%-30% |
| Niacinamide | 2% | 5% | 10% | 0% | 0%-25% |
| Panthenol | 1% | 1% | 3% | 5% | 0.5%-5% |
| Vitamin E | 0.50% | 0.50% | 1% | 2% | 0-2% |
| Acetate | | | | | |
| Methyl paraben | 0.30% | 0.30% | 0.4% | 0.4% | 0.2%-0.5% |
| Lauryl lactate | 1.3% | 1.3% | 4% | 0% | 0%-6.0% |
| Total | 100% | 100% | 100% | 100% | |

Accordingly, in one embodiment, the ratio of water-soluble polymer to water-insoluble polymer is between 0.1-1, more preferably between 0.15-0.95, more preferably between 0.2-0.9, more preferably between 0.2-0.85. In one embodiment, the active agent with activity to improve aesthetic appearance of skin is glycerin and/or water, where glycerin is present in a range of 6-20 wt % of the composition and water is present in an amount between 10-30 wt %. In another embodiment, the solvent mixture in the composition is comprised of water, a polyol and an alpha hydroxyl acid ester or fatty alcohol, and the mixture is present in the composition at between about 10-60 wt %, more preferably between 12-58 wt %, or between 15-55 wt %. In one embodiment, the solvent mixture is comprised of water, glycerin and lauryl lactate, where the lauryl lactate is between 0.1-6 wt %, between 0.2-3 wt %, between 0.5-2 wt %; and where glycerin is present in a range of 6-20 wt % and water is present in a range of 10-30 wt %.

The adhesive layer can be deposited onto a backing layer, or a backing layer may be incorporated into the adhesive layer, thus forming a device for topical application to the skin. The backing layer is a material that is conformable to the body region in which the device is placed, and also has a selected moisture vapor transmission rate that optimizes penetration of the active agent into the skin. Conformable materials generally have some degree of flexibility, and thinner materials are generally more conformable than thicker materials. As thickness of the backing decreases, its MVTR or occlusiveness will typically decrease. Examples of materials considered for use in the device described herein are set forth in Table 2.

TABLE 2

| | | Thickness | MVTR |
|---|---|---|---|
| Brand name | Chemical name | (mil) | (g/m²/day) |
| 3M ™ CoTran ™ 9701 | Polyurethane | 3 | 709 |
| 3M ™ CoTran ™ 9728 | EVA, 18.5% VA | 2 | 87.2 |
| 3M ™ CoTran ™ 9715 | EVA, 18.5% VA | 3 | 64.8 |
| 3M ™ CoTran ™ 9702 | EVA, 9% VA | 2 | 52.8 |
| 3M ™ CoTran ™ 9716 | EVA, 18.5% VA | 4 | 48.6 |
| 3M ™ CoTran ™ 9705 | EVA, 9% VA | 3 | 35.2 |
| 3M ™ CoTran ™ 9706 | EVA, 9% VA | 4 | 26.4 |
| 3M ™ CoTran ™ 9726 | EVA, 2.5% VA | 2 | 12.8 |
| 3M ™ CoTran ™ 9701 | Polyethylene (PE) | 3 | 6 |

*EVA = ethylene-vinyl acetate; VA = vinyl acetate

The device will typically be supplied to an end user with a release line secured to the skin-contacting side of the adhesive mixture that contacts the skin, the release liner being removed by the user prior to use.

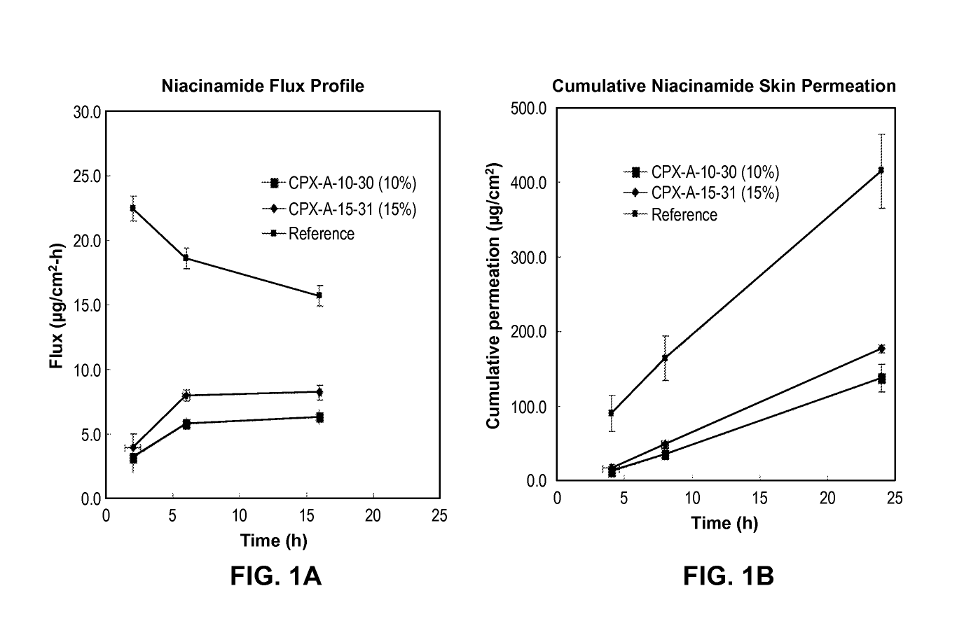

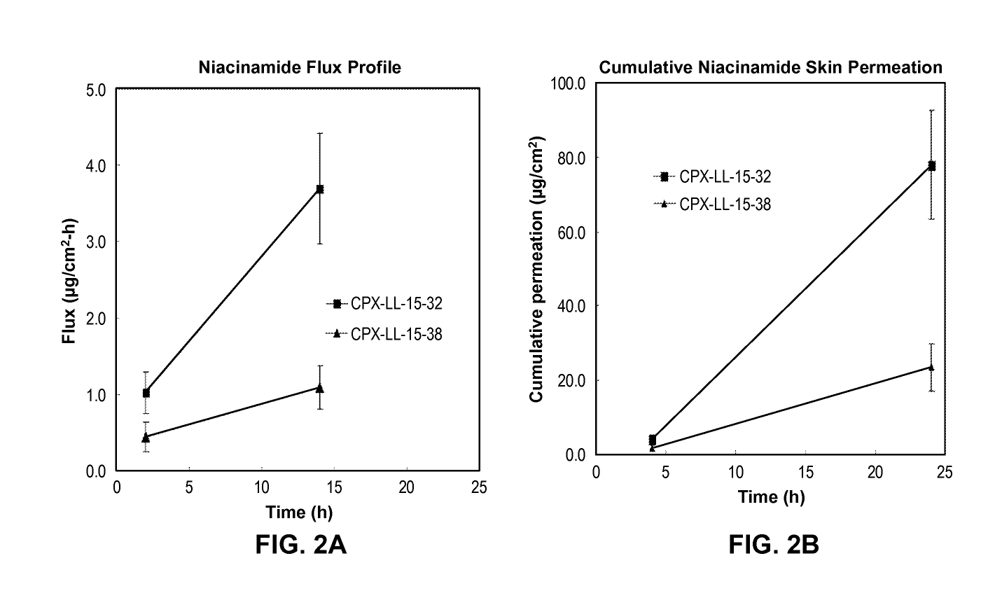

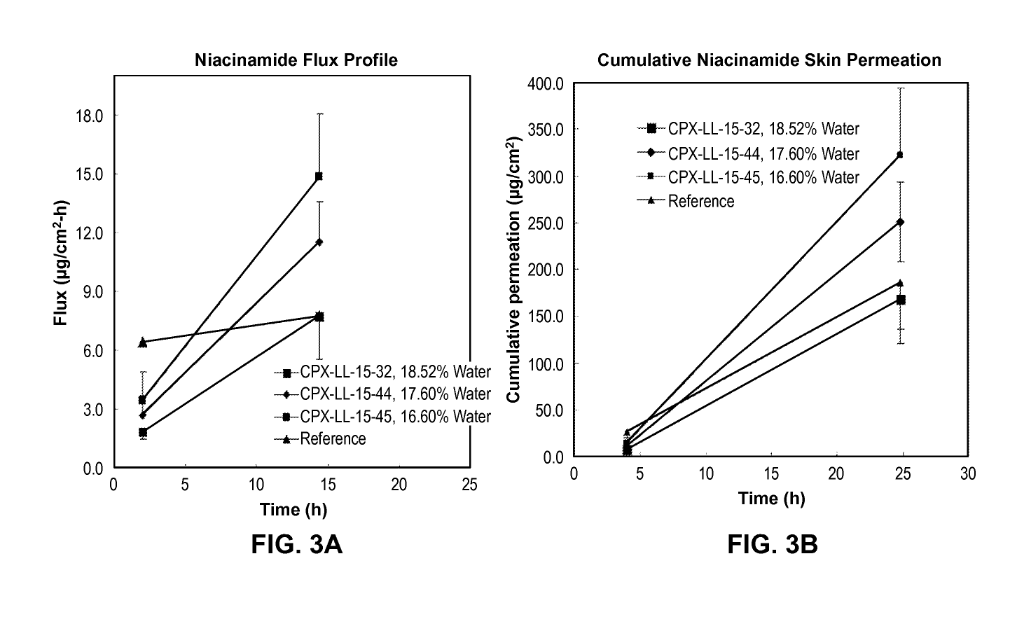

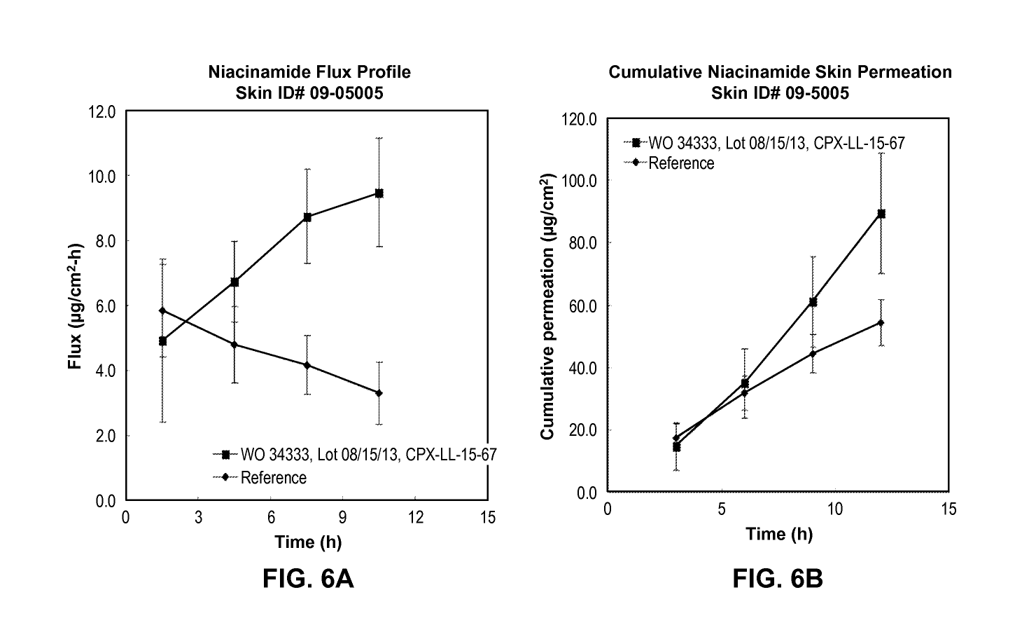

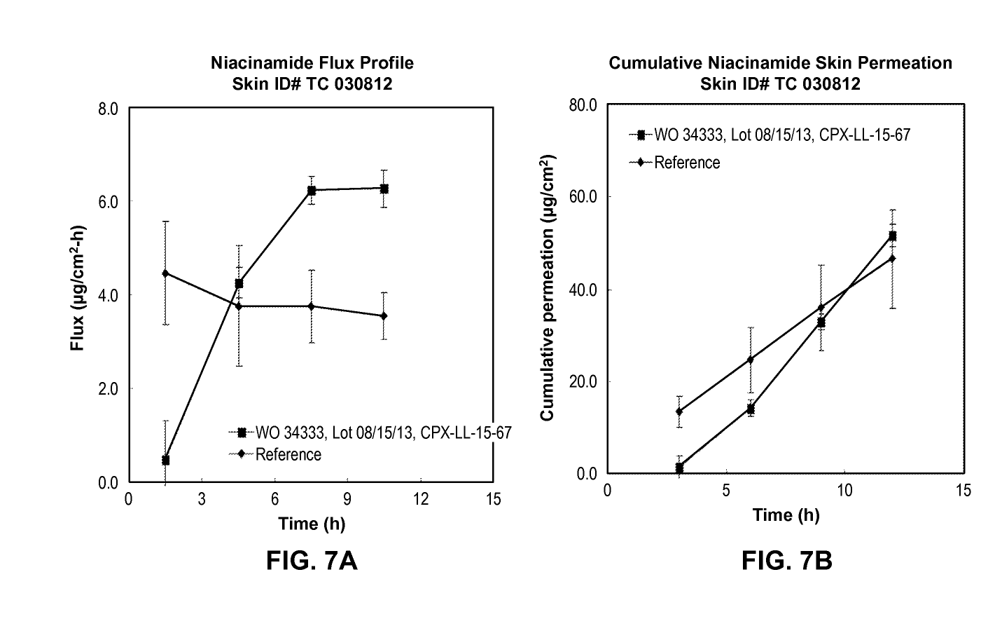

Examples 1-4 set forth an exemplary adhesive compositions and devices. Release of niacinamide from the exemplary compositions and devices was measured in vitro, and is shown in FIGS. 1-7.

In one embodiment, the adhesive mixture further comprises a means to reduce cold flow. In one embodiment, the means comprises adding a fabric or substrate to the adhesive, and examples include nonwoven netting fabrics made of polymeric (thermoplastic) materials (e.g. the non-woven fabric sold under the trade name DELNET®). The fabric is incorporated into the adhesive matrix and serves as a ‘tie’ layer to reduce cold flow of the adhesive matrix. In one embodiment, the fabric is embedded into the matrix. The means to reduce cold flow, in another embodiment, is a seal about the periphery of the adhesive matrix made by application of heat to the periphery. In another embodiment, the means to reduce cold flow comprises a matrix with peripheral edge region that has a thickness less than the non-edge regions of the matrix. In one embodiment, the peripheral edge region has a thickness that is 10%, 20%, 25%, 30%, 40%, or 50% less than the thickness of the adhesive matrix measured at a center point of the matrix. In one embodiment, the peripheral edge region that has a reduced thickness has a width of less than 5 mm, less than 3 mm, or less than 1 mm, and in other embodiments the width of the peripheral edge region with a thickness less than the center of the adhesive matrix is between 0.05-5 mm, 0.1-5 mm, 0.5-5 mm.

In another embodiment, the adhesive mixture comprises a hydrophobic component. The hydrophobic component may be a hydrophobic polymer that when admixed with the adhesive composition described herein above yields an adhesive matrix with a hydrophobic phase and a hydrophilic phase. In one embodiment, the hydrophobic component and the hydrophilic polymers are admixed to form a homogeneous blend that is extruded or cast to form the matrix. The hydrophobic component can be, for example, crosslinked butyl rubbers, wherein a “butyl rubber,” as well known in the art, is an isoprene-isobutylene copolymer typically having an isoprene content in the range of about 0.5 to 3 wt. %, or a vulcanized or modified version thereof, e.g., a halogenated (brominated or chlorinated) butyl rubber. In one embodiment, the hydrophobic component is butyl rubber crosslinked with polyisobutylene. Other suitable hydrophobic polymers include, for example, natural rubber adhesives, vinyl ether polymers, polysiloxanes, polyisoprene, butadiene acrylonitrile rubber, polychloroprene, atactic polypropylene, and ethylene-propylene-diene terpolymers. Other hydrophobic components are described in U.S. Pat. No. 6,803,420, incorporated by reference herein. The definitions of “hydrophobic” and “hydrophilic” polymers are based on the amount of water vapor absorbed by polymers at 100% relative humidity. According to this classification, hydrophobic polymers absorb only up to 1 wt. % water at 100% relative humidity (“rh”), while moderately hydrophilic polymers absorb 1-10 wt. % water, hydrophilic polymers are capable of absorbing more than 10 wt. % of water, and hygroscopic polymers absorb more than 20 wt. % of water.

### III. Methods of Treatment

In another aspect, a method for improving the skin's appearance is provided. In one embodiment, the method comprises topically applying a composition or device that comprises an active agent intended to provide a beneficial effect to reduce fine lines and/or wrinkles, and/or to reduce redness associated with acne lesions and/or protect from formation of acne lesions, and/or to improve skin tone, such as by reducing regions of hyperpigmentation and/or to improve hydration/moisture level in the skin, and/or to improve skin texture or brightness.

a. Method to Reduce Appearance of Fine Lines and Wrinkles

A wrinkle, also known as a rhytide, is a fold, ridge or crease in the skin. Skin wrinkles typically appear as a result of the aging process, and can also be associated with habitual sleeping positions, loss of body mass, or temporarily, as the result of prolonged immersion in water. Age wrinkling in the skin is promoted by habitual facial expressions, aging, sun damage, smoking, poor hydration, and various other factors. Wrinkles associated with aging reflect a change in the skin's structure and functions, and are progressive in that the appearance of fine lines and deep wrinkles increase with age. In one embodiment, a method for reducing the appearance of fine line and/or wrinkles is provided, by providing for topical application an adhesive composition or device as described herein that comprises an active agent effective to reducing the appearance of fine line and/or wrinkles.

In one embodiment, the method comprises topically applying a device or composition that comprises as the active agent niacinamide. In some embodiments, the composition or device further comprises a second agent that has a synergistic effect to increase activity of niacinamide. The second agent may be selected from the group consisting of palmitoyl-lysine-threonine, palmitoyl-lysine-threonine-threonine-lysine-serine, N-undecyl-10-enoyl-L-phenylalanine, retinyl propionate, N-acetyl glucosamine, vitamin C, tretinoin and combinations thereof. One exemplary composition is niacinamide in combination with palmitoyl-lysine-threonine, palmitoyl-lysine-threonine-threonine-lysine-serine, and retinyl propionate.

As illustrated by the exemplary compositions and devices described herein, and the data showing release of niacinamide from the adhesive matrix, skin-contacting layer, the compositions are effective to reduce fine lines and/or wrinkles on human skin. A user topically applies the composition or device to a region on skin where it is desired to improve, by diminishing, the appearance of fine lines or wrinkles. The device is secured to the skin for a period of time, typically for several hours which intends at least 2 hours or more. In one embodiment, a device is applied to the skin for an extended period of at least 6 hours wherein the extended period coincides with sleeping. In another embodiment, a device is applied to the skin, such as a perioribital region, for a period of at least 2-10 hours, preferably 2-8 hours, more preferably 4-10 hours or 4-8 hours.

In one embodiment, the composition or device comprises a backing layer or other layer that renders it occlusive to transmission of water vapor, thus increasing the water content of the skin in contact with the device or composition, to increase skin hydration content relative to the skin hydration content prior to application of the device or composition. Improved hydration of the skin diminishes the appearance of fine lines and wrinkles.

In another embodiment, wearing the device or composition improves the visual appearance of skin by increasing the smoothness, brightness and/or texture of the skin.

The device can be applied one or more times each day. In one embodiment, the device is topically applied once daily for a period of at least 1 month, 2 months, 3 months, 4 months, 6 months, 8 months or 12 months. In one embodiment, the device is applied at least once daily, for a period of 6 hours, on a chronic basis. In one embodiment, the device is applied to an individual of at least 40 years of age, or at least 50 years or age. In other embodiments, the device is applied less than once daily, and is used in combination with a second, different cosmetic therapy.

In other embodiments, the device is applied and worn every other day, every third day, every fourth day, every sixth day, or once weekly. On days when a device or composition is not applied and being worn for a period of time, a different treatment regimen is applied or conducted on the skin. The different treatment regimen may be application of a cosmetic product, such as a cream or ointment, or conducting a non-surgical cosmetic procedure, such as a light or laser treatment, a multi-level skin peel, microdermabrasion, ultrasound or microneedling.

### B. Method to Diminish Regions of Pigmentation

In another embodiment, a method treating hyperpigmentation is provided. Regions of discoloration due to hyperpigmentation are observed in skin, and can be due to aging and/or sun exposure. These regions can be improved or attenuated by providing an agent with skin lightening activity. In some cases, the regions of hyperpigmentation are referred to as age spots.

In one embodiment, a composition or device as described herein is provided for topical application, where the adhesive matrix comprises an agent with activity to lighten skin areas of discoloration and/or is effective to reduce pigmentation associated with age spots and hyperpigmentation. In one embodiment, the active agent is niacinamide. In another embodiment, the active agent is niacinamide in combination with a second agent, such as N-acetyl-glycosamine or vitamin C.

In one embodiment, niacinamide is present in the adhesive composition in an amount between 1-20 wt % of the adhesive composition total weight and N-acetyl-glycosamine or vitamin C is present in an amount between 0.5-10 wt % of the adhesive composition total weight.

In other embodiments, the composition or device is applied to the area of discoloration on the skin daily, typically during a period of sleep, for a period of 2-8 weeks, or 4-16 weeks and/or until there is a reduction in the region of discoloration by at least about 25% or 50% over baseline.

In one embodiment, the method comprises topically applying a device or composition that comprises as the active agent niacinamide. In some embodiments, the composition or device further comprises a second agent that has a synergistic effect to increase activity of niacinamide. The second agent may be selected from N-undecyl-10-enoyl-L-phenylalanine or vitamin C or lectins.

A user topically applies the composition or device to a region on skin where it is desired to improve, by diminishing, the region of hyperpigmentation or discoloration. The device is secured to the skin for a period of time, typically for several hours which intends at least 2 hours or more. In one embodiment, a device is applied to the skin for an extended period of at least 6 hours wherein the extended period coincides with sleeping. In another embodiment, a device is applied to the skin for a period of at least 2-10 hours, preferably 2-8 hours, more preferably 4-10 hours or 4-8 hours. The device can be applied one or more times each day. In one embodiment, the device is topically applied once daily for a period of at least 1 month, 2 months, 3 months, 4 months, 6 months, 8 months or 12 months. In one embodiment, the device is applied at least once daily, for a period of 6 hours, on a chronic basis. In one embodiment, the device is applied to an individual of at least 40 years of age, or at least 50 years or age.

In other embodiments, the device is applied and worn every other day, every third day, every fourth day, every sixth day, or once weekly. On days when a device or composition is not applied and being worn for a period of time, a different treatment regimen is applied or conducted on the skin. The different treatment regimen may be application of a cosmetic product, such as a cream or ointment, or conducting a non-surgical cosmetic procedure, such as a light or laser treatment, a multi-level skin peel, microdermabrasion, ultrasound or microneedling.

### C. Method to Improve Skin Tone

Also contemplated is a method of lightening skin or evening skin tone comprising applying the compositions or devices as described herein, where the adhesive matrix comprises an active agent that can lighten skin or even out pigmentation of the skin. In one embodiment, the method comprises topically applying a device or composition that comprises as the active agent niacinamide. In some embodiments, the composition or device further comprises a second agent that has a synergistic effect to increase activity of niacinamide. The second agent may be selected from N-undecyl-10-enoyl-L-phenylalanine or vitamin C or lectins. In one embodiment, niacinamide is present in an amount between 1-20 wt % of the adhesive composition total weight and N-undecyl-10-enoyl-L-phenylalanine or vitamin C or a lectin is present in an amount between 0.2-10 wt % of the adhesive composition total weight.

A user topically applies the composition or device to a region on skin where it is desired to improve appearance by making the skin tone more uniform, such as by diminishing one or more regions of hyperpigmentation or discoloration. The device is secured to the skin for a period of time, typically for several hours which intends at least 2 hours or more. In one embodiment, a device is applied to the skin for an extended period of at least 6 hours wherein the extended period coincides with sleeping. In another embodiment, a device is applied to the skin for a period of at least 2-10 hours, preferably 2-8 hours, more preferably 4-10 hours or 4-8 hours. The device can be applied one or more times each day. In one embodiment, the device is topically applied once daily for a period of at least 1 month, 2 months, 3 months, 4 months, 6 months, 8 months or 12 months. In one embodiment, the device is applied at least once daily, for a period of 6 hours, on a chronic basis. In one embodiment, the device is applied to an individual of at least 40 years of age, or at least 50 years or age.

In other embodiments, the device is applied and worn every other day, every third day, every fourth day, every sixth day, or once weekly. On days when a device or composition is not applied and being worn for a period of time, a different treatment regimen is applied or conducted on the skin. The different treatment regimen may be application of a cosmetic product, such as a cream or ointment, or conducting a non-surgical cosmetic procedure, such as a light or laser treatment, a multi-level skin peel, microdermabrasion, ultrasound or microneedling.

### D. Method to Improve Appearance of Skin Susceptible to or Effected by Acne Lesions

Also contemplated is a method of treating existing acne lesions, reducing redness associated with acne lesions and/or protecting from formation of acne lesions comprising applying the compositions or devices as described herein, where the adhesive matrix comprises an active agent that treat existing acne lesions, reduce redness associated with acne lesions and/or protect from formation of acne lesions. In one embodiment, the method comprises topically applying a device or composition that comprises as the active agent niacinamide. In some embodiments, the composition or device further comprises a second agent that has a synergistic effect to increase activity of niacinamide. The second agent may be selected from salicylic acid, benzoic acid, benzoyl peroxide, tretinoin. In one embodiment, niacinamide is present in an amount between 1-20 wt % of the adhesive composition total weight and salicylic acid, benzoic acid, benzoyl peroxide, or tretinoin is present in an amount between 0.2-10 wt % of the adhesive composition total weight.

A user topically applies the composition or device to a region on the skin susceptible to acne lesions or where an acne lesion is present. The composition or device is secured to the skin for a period of time, typically for several hours which intends at least 2 hours or more. In one embodiment, a device is applied to the skin for an extended period of at least 6 hours wherein the extended period coincides with sleeping. In another embodiment, a device is applied to the skin for a period of at least 2-10 hours, preferably 2-8 hours, more preferably 4-10 hours or 4-8 hours. The device can be applied one or more times each day. In one embodiment, the device is topically applied once daily for a period of at least 1 month, 2 months, 3 months, 4 months, 6 months, 8 months or 12 months. In one embodiment, the device is applied at least once daily, for a period of 6 hours, on a chronic basis. In other embodiments, the device is applied every other day, every third day, every fourth day, or once a week, and is used in combination with a different, non-surgical cosmetic product or procedure.

In the methods described, the composition or device can be worn during sleep. The method can include topically applying a composition or device to a user's skin prior to the user falling asleep. The method can further include removing the composition or device from the user's skin after the user wakes up. In this way, the treatment occurs at a time that is not disruptive to the user, and achieves the goal of improving visual appearance of the skin, by, for example, reducing the appearance of dark spots or aged spots, evening out the skin's tone, reducing the appearance of fine lines and wrinkles, reducing acne blemishes and associated redness. The composition can be applied during the evening hours prior to falling asleep and can be removed when the user awakens. The composition can be applied to all types of skin such as the face, neck, decolette, arms, hands, body, legs, feet, etc.

The methods described herein may comprise wearing the composition or device for a period of at least about 4 hours, 6 hours, 8 hours, 10 hours, or 12 hours. In still another embodiment, the composition or device is worn while sleeping. The methods may further comprise applying and wearing a device or composition as described herein daily, every other day, every third day, every fourth day, every sixth day, or once weekly. On days when a device or composition is not applied and worn for a period of time, a different treatment regimen is applied or conducted on the skin. The different treatment regimen may be application of a cosmetic product, such as a cream or ointment, or may be conducting a non-surgical cosmetic procedure, such as a light or laser treatment, a multi-level skin peel, microdermabrasion, ultrasound or microneedling.

It will also be appreciated that the devices and compositions described herein provide a method for sparing the cosmetic products and/or procedures a subject requires to achieve an improved appearance of skin. The compositions and devices described herein when applied and worn for a period of time improve retention of active or beneficial agents in the skin. Because the beneficial agents are retained in the skin, less frequent application of the agent or a reduced amount of the agent is needed to achieve the same aesthetic effect in the absence of the compositions or devices described herein.

### IV. Examples

The following examples are illustrative in nature and are in no way intended to be limiting.

### Example 1

### Exemplary Adhesive Composition

An adhesive composition was prepared with the following components.

| | Exemplary | |
|---|---|---|
| Component | Formulation | Component Range |
| Water-soluble polymer- | 25.30% | 20%-35% |
| Polyvinylpyrrolidone, (PVP K90) | | |
| Oligomer-Polyethylene glycol 400 | 12.65% | 6%-20% |
| Water-insoluble polymer- | 12.65% | 6%-20% |
| methacrylic acid-methyl | | |
| methacrylate copolymer | | |
| (EUDRAGIT ® L100-55) | | |
| Glycerin | 12.00% | 6%-20% |
| Water | 18.6% | 10%-25% |
| Niacinamide | 15.00% | 5%-25% |
| Panthenol | 1.00% | 0.5%-5% |
| Vitamin E Acetate | 0.50% | 0-2% |
| Methyl paraben | 0.30% | 0.2%-0.5% |
| Lauryl lactate* | 2.0% | 1.0%-4.0% |
| Total | 100% | |

*Lauryl lactate can be replaced with alpha hydroxyl acid esters or other fatty alcohols such myristyl lactate.

### Example 2

### Exemplary Adhesive Composition

An adhesive composition having the components itemized in Example 1 is prepared, with the following variations: the weight percent of niacinamide is decreased to 2 wt %; the weight percent of lauryl lactate is decreased to 1.33%; the glycerin content is increased to 15 wt % and the water content is adjusted to 29.3 wt %. Release of niacinamide from the compositions is measured using Franz diffusion cells with a cell diffusion area of 0.64 cm² and a human cadaver skin epidermis size of 1.98 cm². The receptor solution is 0.9% NaCl with a volume of 7.5 mL. The test is conducted at 32° C., and the volume of receptor solution is replaced at each sampling time point.

### Example 3

### Adhesive Compositions

Adhesive compositions having the components itemized in Example 1 were prepared, with the following variations: the weight percent of niacinamide was decreased to 10 wt % from 15 wt %; lauryl lactate was replaced with triacetin; the water content was adjusted to be 17.60 wt % or 16.60 wt %. Release of niacinamide from the compositions was measured using Franz diffusion cells with a cell diffusion area of 0.64 cm², a human cadaver skin epidermis size of 1.98 cm². The receptor solution was 0.9% NaCl with a volume of 7.5 mL. The test was conducted at 32° C., and the volume of receptor solution was replaced at each sampling time point. Results are shown in FIGS. 1-3.

### Example 4

### Device with Adhesive Composition and Backing Layer

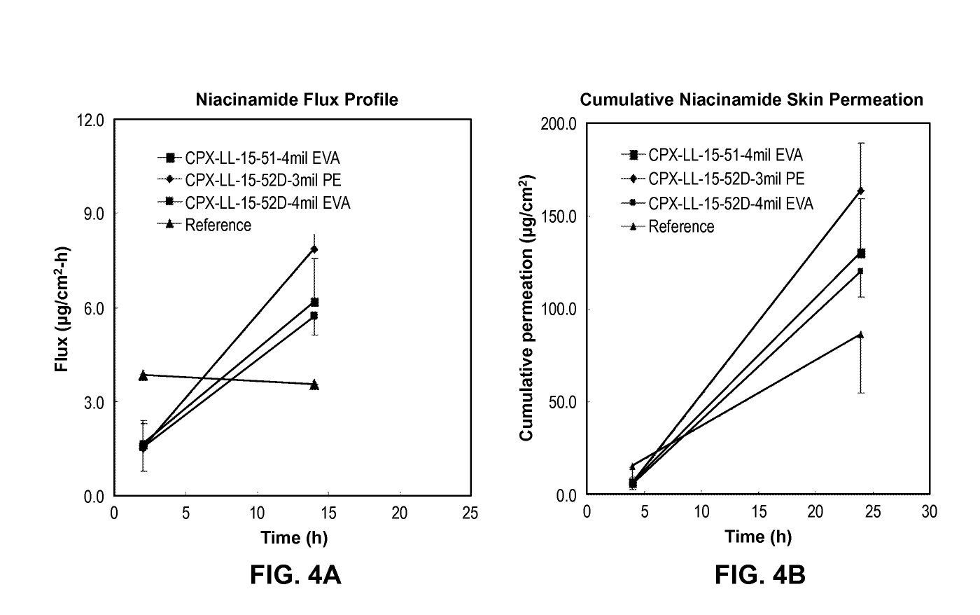

An adhesive composition having the components itemized in Example 1 was prepared and deposited on various backing materials—4 mil ethylene vinyl acetate (EVA) film with 18.5% VA content, a 3 mil polyethylene (PE) film, and a 4 mil EVA film with 9% VA content. Release of niacinamide from the compositions was measured using Franz diffusion cells with a cell diffusion area of 0.64 cm², a human cadaver skin epidermis size of 1.98 cm². The receptor solution was 0.9% NaCl with a volume of 7.5 mL. The test was conducted at 32° C., and the volume of receptor solution was replaced at each sampling time point. Results are shown in FIGS. 4A-4B.

### Example 5

### In Vitro Skin Flux of Niacinamide

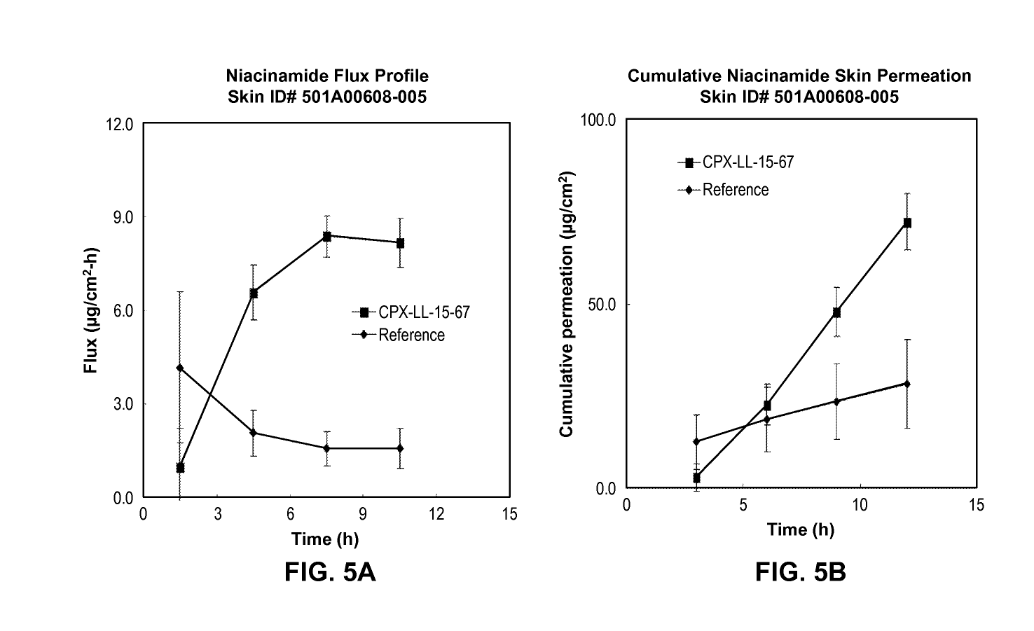

A device comprised of the adhesive layer described in Example 1 and a backing layer of ethylene vinyl acetate, with an 18.5% vinyl acetate content (3M™ CoTran™ 9716) was prepared. Release of niacinamide from the device was evaluated using Franz diffusion cells with a cell diffusion area of 0.64 cm², a human cadaver skin epidermis size of 1.98 cm². The receptor solution was 0.9% NaCl with a volume of 7.5 mL. The test was conducted at 32° C., and the volume of receptor solution was replaced at each sampling time point. Results are shown in FIGS. 5-7.

### Example 6

### Method of Use

A device comprised of an adhesive layer similar to that described in Example 1 but containing in addition to niacinamide palmitoyl-lysine-threonine, palmitoyl-lysine-threonine-threonine-lysine-serine, and retinyl propionate and a backing layer of ethylene vinyl acetate, with an 18.5% vinyl acetate content (3M™ CoTran™ 9716) is prepared.

A controlled study is designed that consists of two months of device use. The test site is the face of selected female panelists. The women are instructed to refrain from using any treatment products on the test site during the test period except for the test devices provided. Skin evaluations are carried out before treatment (baseline), and two, four, and eight weeks during the course of treatment. The women are instructed to apply 2 devices to each side of the face once a day in the evening before sleep. The device is pressed firmly onto the skin for 15 to 30 seconds. Device use is monitored by a daily diary as well as assessment of remaining package content at the end of the study.

Skin Tone Study: At the outset of the study, a particular area to measure skin tone on the cheeks of each panelist is marked. The images of that specific portion of the face are obtained using a fiber optic microscope (Scalar, Vacaville, Calif.) at a resolution of 640×320 (approximately 1 cm²). Three images are recorded from each cheek. The same area is photographed at each time point following the initial visit. The stored images are digitized and analyzed using an image analysis program, Optimas 6.51. The standard deviation of the average Grey value of each of the three color channels is determined. This is a measure of the amount of variation in the picture in terms of color. If a product has been effective in evening skin color there will be a decrease in variation and a concomitant decrease in the variance of the Grey value.

Age Spot Study: At the outset of the study, a particular area demonstrating age spots on the cheeks or hand of each panelist is marked. The images of that specific portion of the face or hand are obtained using a fiber optic microscope (Hi-Scope) at a magnification of 20 times. Three age spots are chosen per panelist. The same area is photographed at each time point following the initial visit. The stored RGB images are digitized and analyzed using an image analysis program, Optimas 6.51. The stored images are digitized and analyzed to determine the average Grey value (i.e. density) and area of the corresponding age spot. If a product is effective in diminishing the appearance of age spots, an increase in Grey Value (density) and a decrease in spot area will occur.

Skin Whitening Study: Skin whitening is assessed and documented with close up photography. Photos of the right and left facial cheeks are taken with a Nikon M3 digital camera. Panelists heads are placed in a head rest to insure reproducibility of positioning. The camera is positioned two feet from the panelist at an F stop of 32. A crossed polarized lens is used to remove all glare from the photographs. Photos are evaluated via an image analysis program, Optimas 6.51, comparing before and after product use. Whitening is analyzed by determining the average Grey value of the three color channels (RGB) in each photograph. If the product has been effective in whitening skin there will be an increase in the Grey value.

The device significantly (p<0.05) improves skin tone, as compared to the baseline measurement, by an average of 25%, reduced the appearance of age spots by an average of 25%, and improved skin whitening by an average of 25% after 8 weeks of product use as compared to pre-treatment.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A topical skin composition, comprising:
a skin-contacting layer comprised of (i) an adhesive mixture comprised of a water-swellable, water-insoluble film forming polymer and a water-soluble film forming polymer in a ratio of between about 0.1-1.0; (ii) between about 15-55 wt % of a solvent mixture comprised of water, a polyol and an alpha hydroxyl acid ester or fatty alcohol; and (iii) between about 1-50 wt % of an active agent for improving aesthetic appearance of skin.

2. The composition of claim 1, wherein the adhesive mixture further comprises an oligomer capable of hydrogen or electrostatic bonding to the water-soluble film forming polymer.

3. The composition of claim 1, wherein the water-soluble film forming polymer and the oligomer form a homogenous blend.

4. The composition of claim 1, wherein the water-swellable, water-insoluble film forming polymer is a cellulose ester or an acrylate polymer.

5. The composition claim 4, wherein the acrylate polymer is selected from polymers and copolymers of acrylic acid, methacrylic acid, methyl acrylate, ethyl acrylate, methyl methacrylate, or ethyl methacrylate.

6. The composition of claim 1, wherein the water-soluble film forming polymer is selected from poly(N-vinyllactams), poly(N-vinyl amides), poly(N-alkylacrylamides), polyacrylic acids, polymethacrylic acids, polyvinyl alcohol, polyvinylamine, and copolymers and blends thereof.

7. The composition of claim 5, wherein in the oligomer is selected from polyhydric alcohols, monomeric and oligomeric alkylene glycols, polyalkylene glycols, carboxyl-terminated polyalkylene glycols, amino-terminated polyalkylene glycols, ether alcohols, alkane diols and carbonic diacids.

8. The composition of claim 1, further comprising a hydrophobic adhesive layer disposed between the backing layer and the adhesive layer.

9. The composition of claim 1, wherein the active agent is capable of reducing visual appearance of fine lines and/or wrinkles.

10. The composition of claim 9, wherein the active agent is niacinamide.

11. The composition of claim 10, further comprising a second agent that has a synergistic effect to increase activity of niacinamide.

12. The composition of claim 11, wherein the second agent is selected from the group consisting of palmitoyl-lysine-threonine, palmitoyl-lysine-threonine-threonine-lysine-serine, N-undecyl-10-enoyl-L-phenylalanine, retinyl propionate, N-acetyl glucosamine, vitamin C, tretinoin and combinations thereof.

13. The composition of claim 11, wherein the second agent is a combination of palmitoyl-lysine-threonine, palmitoyl-lysine-threonine-threonine-lysine-serine, and retinyl propionate.

14. The composition of claim 1, wherein the active agent is effective to treat existing acne lesions, reduce redness associated with acne lesions and/or protect from formation of acne lesions.

15. The composition of claim 14, wherein the active agent is selected from niacinamide, salicylic acid, benzoic acid, benzoyl peroxide, tretinoin, and combinations thereof.

16. The composition of claim 1, wherein the active agent is effective to reduce pigmentation associated with age spots and hyperpigmentation.

17. The composition of claim 16, wherein the active agent is niacinamide.

18. The composition of claim 17, further comprising N-acetyl-glycosamine.

19. The composition of 18, wherein niacinamide is present in an amount between 1-20 wt % of the adhesive composition total weight and N-acetyl-glycosamine or vitamin C is present in an amount between 0.5-10 wt % of the adhesive composition total weight.

20. The composition of claim 1, wherein the active agent is effective to improve an uneven skin tone by diminishing areas of hyperpigmentation and/or hypopigmentation.

21. The composition of claim 20, wherein the active agent is niacinamide.

22. The composition of claim 21, further comprising N-undecyl-10-enoyl-L-phenylalanine or vitamin C or lectins.

23. The composition of 22, wherein niacinamide is present in an amount between 1-20 wt % of the adhesive composition total weight and N-undecyl-10-enoyl-L-phenylalanine or vitamin C or a lectin is present in an amount between 0.2-10 wt % of the adhesive composition total weight.

24. A method for reducing fine lines and/or wrinkles on human skin, comprising:
topically applying an adhesive composition of claim 1.

25. A method for treating existing acne lesions, reducing redness associated with acne lesions and/or protecting from formation of acne lesions, comprising:
topically applying an adhesive composition of claim 1.

26. A method for improving skin tone, such as by reducing regions of hyperpigmentation such as those often referred to as age spots or by reducing regions of discoloration associated with aging skin's exposure to sun, comprising:
topically applying an adhesive composition of claim 1.

27. A method for improving the aesthetic appearance of skin comprising:
topically applying an adhesive composition of claim 1.

28. The method of claim 24, further comprising wearing the composition or device while sleeping.

29. The method of claim 24, wherein said topically applying comprises topically applying to the face.

30. The method of claim 24, wherein said topically applying comprises topically applying in the under-eye region of the face.

31. The method of claim 24, wherein said topically applying comprises topically applying in a region around the eye.

32. A device for topical application to the skin, comprising:
a backing layer;
a skin-contacting layer deposited on the backing layer, the skin-contacting layer comprised of
(i) an adhesive mixture comprised of a water-swellable, water-insoluble film forming polymer and a water-soluble film forming polymer in a ratio of between about 0.1-01.0;
(ii) between about 15-55 wt % of a solvent mixture comprised of water, a polyol and an alpha hydroxyl acid ester or fatty alcohol, the solvent mixture in liquid form at room temperature; and
(iii) between about 1-50 wt % of an active agent for improving aesthetic appearance of skin.
